(19) United States
(12) Patent Application Publication
(10) Pub. No.: US 2002/0095205 A1
(43) Pub. Date: Jul. 18, 2002

(54) Encapsulated radiopaque markers

(76) Inventors: Tarun J. Edwin, Chandler, AZ (US); Roberta L. Druyor-Sanchez, Mesa, AZ (US)

Correspondence Address: Todd W. Wight, Morrison & Foerster LLP, 555 West Fifth Street, Los Angeles, CA, 90013-1024, US

(21) Appl. No.: 09/760,254

(22) Filed: Jan. 12, 2001

(51) Int. Cl. A61F 2/06

(52) U.S. Cl. 623/1.13; 600/431

(57) ABSTRACT

A radiopaque marker that is incorporated into an implantable biocompatible device for precise imaging as the device is delivered and deployed within a body vessel. The radiopaque marker can take on a variety of forms which can be excised from a thin foil made of radiopaque metal or from an ePTFE sheet that has been coated on one or both surfaces with a radiopaque metal. The radiopaque markers, in forms such as rings, strips, disks, rectangles or spheres are encapsulated or contained within the implantable device to prevent the radiopaque metal from dissolving or escaping into the blood stream. Strategic placement of the radiopaque markers at each end of the implantable device enables the physician to fluoroscopically view its exact location prior to deployment and in subsequent follow-up examinations.

### BACKGROUND OF THE INVENTION

1. Field of the Invention

The present invention relates generally to medical devices, and more particularly to a locating marker for implantable biocompatible devices.

2. Description of Related Art

Stents, artificial grafts, and related endoluminal devices are currently used by medical practitioners to treat tubular body vessels or ducts that become so narrowed (stenosed) that flow of blood or other biological fluids is restricted. Such narrowing (stenosis) occurs, for example, as a result of the disease process known as arteriosclerosis. While stents are most often used to “prop open” blood vessels, they can also be used to reinforce collapsed or narrowed tubular structures in the respiratory system, the reproductive system, bile or liver ducts or any other tubular body structure.

Vascular grafts made of polytetrafluoroethylene (PTFE) are typically used to replace or repair damaged or occluded blood vessels within the body. However, they may require additional means for anchoring the graft within the blood vessel, such as sutures, clamps, or similarly functioning elements to overcome retraction. Stents have been used in combination with grafts to provide endovascular prostheses which are capable of maintaining their fit against blood vessel walls. The use of grafts along with stents also serves to overcome a problem found with stents where smooth muscle cells and other tissues can grow through the stent's mesh-like openings, resulting in restenosis of the vessel.

Polytetrafluoroethylene (PTFE) has proven unusually advantageous as a material from which to fabricate blood vessel grafts or prostheses, because PTFE is extremely biocompatible, causing little or no immunogenic reaction when placed within the human body. In its preferred form, expanded PTFE (ePTFE), the material is light, porous and readily colonized by living cells so that it becomes a permanent part of the body. The process of making ePTFE of vascular graft grade is well known to one of ordinary skill in the art. Suffice it to say that the critical step in this process is the expansion of PTFE into ePTFE. This expansion represents a controlled longitudinal stretching in which the PTFE is stretched to several hundred percent of its original length.

The field of covering stents with polymeric coatings and ePTFE in particular has been substantially explored by those skilled in the art. One popular way of covering the stent with ePTFE material is to encapsulate it within two layers of ePTFE, which are subsequently fused together by heat in places where the two layers are in contact through openings in the stent wall. This provides a solid one-piece device that can be expanded and contracted without an ePTFE layer delaminating.

Implantation of a graft or an encapsulated stent into the vasculature of a patient involves very precise techniques. Generally, the device is guided to the diseased or damaged portion of a blood vessel via an implantation apparatus that deploys the graft or the encapsulated stent at the desired location. In order to pinpoint the location during deployment, the medical specialist will generally utilize a fluoroscope to observe the deployment by means of X rays. Deployment of an encapsulated stent at an unintended location can result in immediate trauma, as well as increasing the invasiveness associated with multiple deployment attempts and/or relocation of a deployed device. In addition, visualization of the implanted device is essential for follow-up inspection and treatment. However, in order to implant the encapsulated stent using fluoroscopy, some portion of the stent, graft or implantation device must be radiopaque. This becomes somewhat of a problem due to the fact that many radiopaque metals, which are extremely toxic, may leach out into the blood stream and come into direct contact with portions of the body.

Toxicity is generally not found to be a problem for stents that are expanded within the vessel using a balloon catheter because a balloon catheter apparatus can have radiopaque features incorporated therein. Because the balloon catheter apparatus is inside of the encapsulated stent device during delivery and deployment, and is generally protected from the body upon removal, the radiopaque portions do not make direct contact with the patient's body. However, if the balloon moves after expansion of the stent, the correct placement cannot be confirmed. On the other hand, a graft or a self-expanding stent is generally delivered to the damaged or diseased site via a constraining member in the form of a catheter or sheath and is deployed by removing the constraining member. Thus, in order to direct the device to the precise location for deployment, the radiopacity must be incorporated into the device or the constraining member to confirm the correct placement within the vessel.

The locating of implantable devices utilizing radiopaque markers is well-known in the art. For example, U.S. Pat. No. 5,713,853 to Clark et al. discloses the use of a radiopaque band to assist in the tracking of a catheter. The band is made of radiopaque metal and is placed around the outside of the distal end of the catheter. While the band of Clark et al. may be useful for locating the end of the catheter, it is placed on the outside of the catheter, which may result in toxicity problems. In addition, because the band is solid, it cannot be used in a graft or an encapsulated stent device because it is not flexible and thus cannot expand and contract with the device. Other prior art in the field of locating implantable devices have not addressed these issues.

Therefore, there exists a need to provide a radiopaque marker for incorporation into an implantable biocompatible device that does not come into direct contact with the body, and also allows the device to contract and expand without interference as it is delivered and deployed within a blood vessel of a patient.

### SUMMARY OF THE INVENTION

Accordingly, the present invention provides a radiopaque marker that is incorporated into an implantable biocompatible device so that it can be precisely imaged as it is delivered and deployed within a body vessel. In a preferred embodiment of the present invention, a plurality of thin radiopaque markers are incorporated into an implantable device by encapsulating them between at least two layers of biocompatible material. The radiopaque marker can take on a variety of forms which can be excised from a thin foil made of radiopaque metal or from an ePTFE sheet or structure that has been coated on one or both surfaces with a radiopaque metal. The radiopaque markers, in forms such as rings, strips or disks, are encapsulated or contained within the device to prevent the radiopaque metal from dissolving or escaping into the blood stream. Importantly, the stent itself cannot be coated with radiopaque metal as the metal can interfere with the stent's self-expanding or other metallic properties. Strategic placement of the radiopaque markers at each end of the implantable device enables the physician to fluoroscopically view its exact location prior to deployment and subsequently in follow-up examinations to ensure placement and to verify that no migration has occurred.

The radiopaque coating onto an ePTFE sheet or structure can be accomplished using a vacuum deposition process such as sputtering or electron beam evaporation or by using metal plating procedures. Factors that are important in the composition of the ePFTE embodiment of the radiopaque marker include the temperature at which the radiopaque metal is deposited onto the ePTFE, the metal's ability to adhere to the surface of the ePTFE and the amount of the metal that is deposited thereon. Variations to this embodiment include the specific radiopaque metal used (gold, platinum, iridium, palladium, rhodium, titanium, tungsten, etc.), the type of biocompatible material to be coated (polyester, polyurethanes, plastics, etc.) and the form of the radiopaque marker (sutures, threads, strips, rings, dots, etc.).

These and other features and advantages of the present invention will become more apparent to those skilled in the art when taken with reference to the following more detailed description of the preferred embodiments of the invention and the accompanying drawings.

### BRIEF DESCRIPTION OF THE DRAWINGS

FIG. 1 is a longitudinal view of a partially coated tubular graft structure.

FIG. 2 shows a ring cut from the coated portion of the tubular structure in FIG. 1.

FIG. 3 shows a cut away view of an encapsulated stent device of the present invention with a radiopaque marker near a distal end.

FIG. 4 shows a cut away view of an encapsulated stent device of the present invention with multiple radiopaque markers disposed along the length of the device.

FIG. 5 shows a side view of a partially encapsulated stent with radiopaque markers designating each end of the encapsulated section.

### DETAILED DESCRIPTION OF THE INVENTION

The present invention satisfies the need for a radiopaque marker that can be encapsulated in a graft or along with a self-expanding stent to permit a physician to view the exact location of the device during delivery and deployment thereof. In the detailed description that follows, it should be appreciated that like reference numerals are used to describe like elements illustrated in one or more of the figures.

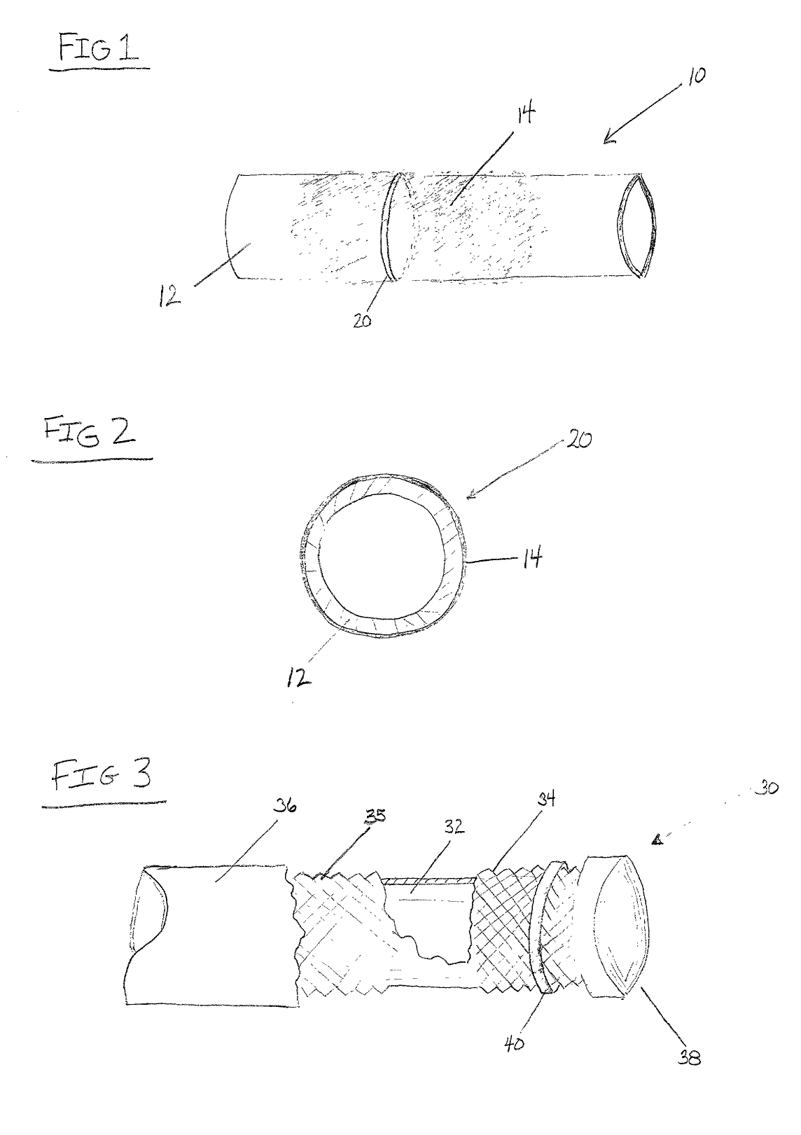

Referring now to FIG. 1, a tubular graft structure 10 is shown. The tubular graft structure 10 includes a graft 12 and a radiopaque coating 14. The graft 10 can be made of a variety of biocompatible materials including polyester and any number of organic plastic polymers including polyurethane, polyester, polyamide and other “plastics;” however, the preferred embodiment of the present invention uses ePTFE. The radiopaque coating 14, which in the preferred embodiment is gold, but could be any number of metals including platinum, iridium, palladium, rhodium, titanium and tungsten, is applied to the graft 12 using either a vacuum deposition process such as sputtering or electron beam evaporation or by using metal plating procedures. As one skilled in the art can appreciate, a coated sheet of ePTFE would produce substantially similar results. The deposition process must be performed at a sufficiently high temperature to ensure bonding between the deposited metal and the graft material. In the preferred embodiment, a temperature above 140° F. was found to provide optimal conditions for bonding. Moreover, it is important that a suitable amount of radiopaque metal be applied to the graft 12 or sheet of ePTFE so that a marker procured therefrom will be visible under fluoroscopy. Of course, the amount of radiopaque metal necessary for fluoroscopic visualization is variable depending on the application of the device to which the locating marker is incorporated. For instance, a locating marker incorporated into a device for repairing an abdominal aortic aneurysm will require a greater amount of radiopaque metal for fluroscopic visualization than one incorporated in a device for more superficial vascular applications. However, in most situations that were tested, the thickness of the coating layer or radiopaque foil must be at least 0.004 in. or the equivalent density to provide fluoroscopic visualization.

The radiopaque locating marker of the present invention can be in many shapes and forms. For instance, as seen in FIG. 1, a ring portion 20 can be taken from the coated section of the tubular graft structure 10. The ring portion 20 is shown in cross-section in FIG. 2 in an enlarged view, illustrating the radiopaque coating 14 circumferentially layered around graft 12. The radiopaque locating marker can also be in the form of any length of strip taken from either the tubular graft structure 10 or a similarly coated ePTFE sheet. The strip can be relatively short, to be placed partially around the circumference of a tubular structure in which it is incorporated (see FIG. 4), or long, in which case it could be placed longitudinally within the device or wrapped around all or a portion of the device in a spiral configuration.

Other forms of the locating marker include sutures, threads and other small pieces such as disks. In particular, one alternate embodiment consists of a radiopaque liquid or paste, such as barium sulfate, that is incorporated into the stent-graft by enclosing it within the graft material. The radiopaque substance could be placed within a designated non-porous pocket within the graft to prevent the substance from leaking. Another alternate embodiment consists of a sphere of non-porous material containing within it a radiopaque substance. This radiopaque sphere is then encapsulated within the graft material. Certainly, it should be appreciated that additional forms not specifically mentioned herein would be included within the spirit and scope of the present invention. It should also be noted that several of these forms could be used in combination to enhance the visualization of the implanted device. Of course, also within the spirit of the invention is an embodiment wherein a section or sections of the encapsulated portion of an ePTFE graft structure is coated with a radiopaque metal. More specifically, in a graft structure containing at least two layers of ePTFE, some or all of the outer surface of a luminal graft layer and the inner surface of an abluminal graft layer are coated with a radiopaque metal before combining the two layers. These layers could be the sole layers of the graft structure or could incorporate a stent or other structure therebetween provided that the radiopaque metal is contained within the graft structure to avoid possible leakage of the metal into the body of a patient.

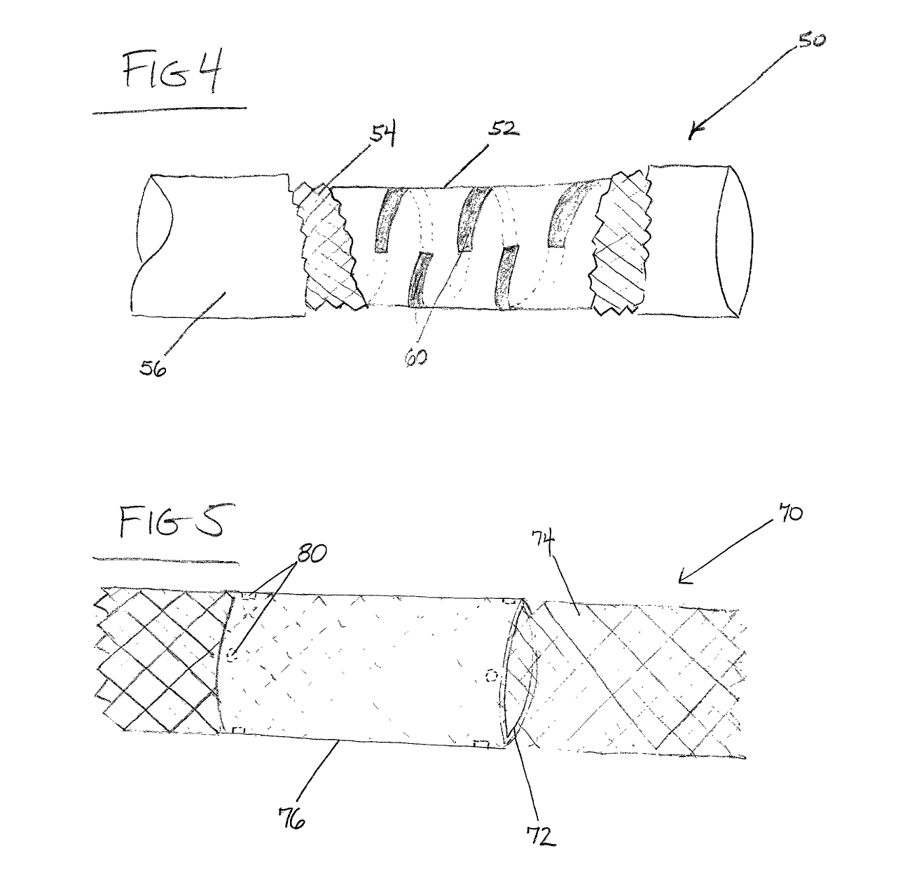

FIG. 3 illustrates an encapsulated stent device 30 in a cut-away view so that all aspects of the device 30 can be seen. An inner tubular ePTFE graft 32 is within a self-expanding stent 34, covering a luminal surface of the stent 34. An abluminal layer 35 of the stent 34 is covered by an outer tubular ePTFE graft 36. Near a distal end 38 of the encapsulated stent device 30, a radiopaque marker 40 is placed around the abluminal layer of the stent 34, but within the outer tubular ePTFE graft 36. The marker 40 allows precision placement of the encapsulated stent device 30 because it enables portions of the device 30 to be viewed using fluoroscopy, thus optimizing delivery and deployment. The radiopaque marker 40 is in the shape of a ring and is made of gold-coated ePTFE so that expansion and contraction of the device is permitted. Although only a distal end 38 of the encapsulated stent device 30 can be seen in FIG. 3, a radiopaque ring 40 is also positioned near a proximal end of the encapsulated stent device 30 so that both ends of the device can be viewed. Optimally, the rings will be placed at the distal and proximal ends of the stent device 30 so that the exact location of both ends can be pinpointed. Of course, any number of radiopaque rings or other locating markers can be included in any arrangement that aids the physician in the deployment process as well as post-operative procedures.

FIG. 4 illustrates an alternate embodiment of the present invention, showing a cut-away view of an encapsulated stent device 50. The stent device 50 includes an outer layer of biocompatible tubular material 56 (preferably ePTFE) that encapsulates a metal support 54, such as a stent, by binding to the inner tubular layer 52. In this embodiment, the inner tubular layer 52, also preferably made of ePTFE, is left unsintered and is therefore soft and sticky. Radiopaque strips 60 that have been produced independently or harvested from an ePTFE structure that has been coated with radiopaque metal, are positioned on top of the unsintered inner tubular layer 52 before the metal support 54 is placed thereon. Because of the sticky properties of the inner tubular layer 52, the radiopaque ePTFE strips 60 easily adhere to its outer surface. As seen in FIG. 4, the strips 60 are arranged circumferentially and are offset an equal distance, resulting in multiple strips evenly spaced apart in two sets, each set covering half of the inner tubular layer 52.

FIG. 5 illustrates yet another embodiment of the present invention. In device 70, a stent 74 is left uncovered on both ends so that only a middle portion of the stent 74 is encapsulated. At each end where the encapsulation portion terminates, radiopaque markers 80 in the form of disks are positioned at 90° intervals around the circumference of the inner tubular layer 72 so that at least two disks can be seen in any two-dimensional plane to enable the physician to identify the end of the ePTFE. Thereby the physician can ensure that side branches/ducts are not occluded or blocked by the biocompatible covering.

At least some portion of the disks 80 are composed of radiopaque metal. In the case of radiopaque-coated ePTFE disks, a portion of the disks 80 have a radiopaque metal incoroporated thereon. On the other hand, the disks 80 can be composed entirely of radiopaque metal, such as disks made of thin radiopaque foil. The radiopaque disks 80 can be placed directly onto the unsintered inner tubular layer 72 for maximum adhesion. As shown in FIG. 5, the disks 80 are positioned to be within a diamond of the stent 74. It should be appreciated that because the disks are so located, they can be placed onto the inner tubular layer 72 either before or after the stent 74 is assembled thereon. In addition it is important that the size of the disk 80 be carefully monitored so as not to interfere with the expansion and contraction of the device 70. Finally, it will be appreciated by those of skill in the art that a radiopaque marker made either partially or entirely of a radiopaque metal can be stratigically placed along the length and/or around the circumference of an implantable device to optimize the fluoroscopic visualization thereof.

Many alterations and modifications may be made by those having ordinary skill in the art without departing from the spirit and scope of the present invention. For example, a radiopaque marker has been illustrated within an encapsulated stent device so that the device can be seen fluoroscopically during implantation. It should be apparent, however, that the inventive concepts described above would be equally germane in other applications where radiopaque markers can be imbedded into implantable devices for locating purposes. Moreover, the words used in this specification to describe the invention and its various embodiments are to be understood not only in the sense of their commonly defined meanings, but to include by special definition in this specification structure, material or acts beyond the scope of the commonly defined meanings. Thus, if an element can be understood in the context of this specification as including more than one meaning, then its use in a claim must be understood as being generic to all possible meanings supported by the specification and by the word itself. The definitions of the words or elements of the following claims are, therefore, defined in this specification to include not only the combination of elements which are literally set forth, but all equivalent structure, material or acts for performing substantially the same function in substantially the same way to obtain substantially the same result.

We claim:

1. A radiopaque locating marker for fluoroscopic visualization, wherein the marker is entirely contained within an implantable device, comprising
a member made of a pliable biocompatible material; and
a radiopaque metal incorporated onto or into the member.

2. The radiopaque locating marker of claim 1, wherein the pliable biocompatible material is expanded polytetrafluoroethylene.

3. The radiopaque locating marker of claim 1, wherein the radiopaque metal is selected from the group consisting of gold, platinum, iridium, palladium, rhodium, titanium and tungsten.

4. The radiopaque locating marker of claim 1, wherein a layer of the metal is deposited on at least one surface of the member, the member having a form selected from the group consisting of a ring, a strip, a disk, a rectangle and a sphere.

5. The radiopaque locating marker of claim 4, wherein a thickness of the layer is greater than 0.004 in.

6. The radiopaque locating marker of claim 1, wherein the member is a non-porous three-dimensional object enclosing the radiopaque metal.

7. A method for making a locating marker for fluoroscopic visualization, comprising the steps of:
depositing a layer of radiopaque metal on at least one surface of a pliable biocompatible material, wherein the layer is of sufficient thickness or density to be viewed fluoroscopically when implanted within a patient; and
cutting the layered pliable biocompatible material into individual pieces.

8. The method of claim 7, wherein the pliable biocompatible material is expanded polytetrafluoroethylene.

9. The method of claim 7, wherein a thickness of the layer is greater than 0.004 in.

10. The method of claim 7, wherein the depositing step further comprises using a process selected from the group consisting of electron beam evaporation, sputtering and metal plating.

11. An implantable biocompatible device for fluoroscopic visualization, comprising:
a tubular radially expandable support member having a plurality of openings passing through walls of the support member,
an expanded polytetrafluoroethylene tubular member, including a luminal and an abluminal layer bonded together, circumferentially surrounding and encapsulating a portion of the support member, wherein at least one end of the support member is bare; and
at least one radiopaque locating marker disposed at each terminal end of the encapsulated portion of the implantable biocompatible device, wherein the at least one radiopaque locating marker is contained within the expanded polytetrafluoroethylene tubular member.

12. The implantable biocompatible device of claim 11, wherein the at least one radiopaque locating marker comprises a combination of an expanded polytetrafluoroethylene member and a radiopaque metal.

13. The implantable biocompatible device of claim 12, wherein the radiopaque metal is selected from the group consisting of gold, platinum, iridium, palladium, rhodium, titanium and tungsten.

14. The implantable biocompatible device of claim 12, wherein the at member has a form selected from the group consisting of a ring, a strip, a disk, a rectangle and a sphere.

15. The implantable biocompatible device of claim 12, wherein the at least one radiopaque locating marker further comprises eight small disks, wherein four disks are circumferentially positioned at each terminal end of the encapsulated portion at 90° intervals and do not come in contact with the support structure.

16. A method for making an endoluminal graft structure for fluoroscopic visualization, the graft structure including at least two ePTFE tubes, comprising the steps of:
depositing a layer of radiopaque metal on a portion of the outer surface of a first ePTFE tube;
depositing a layer of radiopaque metal on a portion of the inner surface of a second ePTFE tube having an inner diameter greater than the outer diameter of the first ePTFE tube, wherein the layers of radiopaque metal deposited on the first and second ePTFE tubes are of sufficient thickness or density to be viewed fluoroscopically when the tubes are implanted within a patient;
positioning the second ePTFE tube over the first ePTFE tube; and
combining the first and second ePTFE tubes, wherein the layers of radiopaque metal are completely contained therein.

17. The method of claim 16, further comprising a step of positioning a support structure between the first and second ePTFE tubes, wherein the combining step includes encapsulation of the support structure.
